(19) United States
(12) Patent Application Publication
(10) Pub. No.: US 2026/0158490 A1
(43) Pub. Date: Jun. 11, 2026

(54) MODULAR MULTI-ANALYTE DIAGNOSTIC TEST SYSTEM

(71) Applicant: Trividia Health, Inc., Fort Lauderdale, FL (US)

(72) Inventors: Brent Modzelewski, Delray Beach, FL (US); Steven Leone, Lake Worth, FL (US); Jose A. Rios Rodriguez, Miami, FL (US); Yitzhak Haim, Fort Lauderdale, FL (US); Edward Cardello, Wellington, FL (US)

(73) Assignee: Trividia Health, Inc., Fort Lauderdale, FL (US)

(21) Appl. No.: 19/416,735

(22) Filed: Dec. 11, 2025

Related U.S. Application Data
(60) Provisional application No. 63/730,599, filed on Dec. 11, 2024.

(51) Int. Cl. B01L 3/00

(52) U.S. Cl. CPC B01L 3/502715; B01L 2200/028; B01L 2300/023; B01L 2300/025

(57) ABSTRACT

Systems and methods are provided for performing multi-analyte testing. In some embodiments, an analyte testing device is provided that includes a plurality of modules, each module of the plurality of modules configured to perform one or more of a plurality of different analyte tests. Each module of the plurality of modules includes at least one sample port for receiving a sample, and at least one microprocessor configured to calculate an amount of one or more different analytes in the sample and prepare test results based on the calculated one or more different analytes. A base unit is provided having a plurality of receptacles configured to receive the plurality of modules such that an electrical connection is established therebetween. Each module of the plurality of modules is configured to utilize the electrical connection to communicate the test results from each module to a processor of the base unit.

### RELATED APPLICATIONS

This application claims the benefit of and priority to U.S. Provisional Application No. 63/730,599 filed Dec. 11, 2024, the contents of which is incorporated herein by reference in its entirety.

### FIELD

This disclosure relates to processes and systems for performing multiple analyte tests using a diagnostic testing device.

### BACKGROUND

Current state of the art for multi-analyte testing falls into two main categories. First, laboratory instruments, usually bench-top, AC powered can accept a multi-test array in the form of a cartridge, where the cartridge contains either a single analyte test for multiple different blood samples or a testing panel of multiple analyte tests. The cartridges are large, expensive, need large blood volumes, generate non-trivial amount of biological refuge and require a trained health care provider to operate them.

Second, smaller hand-held devices with a singular strip port that can run a variety of different analyte tests with simple test strips. The use of these types of devices are limited as they can only accept a single size and configuration of test strip, and typically handle only relatively few different analyte tests.

### SUMMARY

The present disclosure is directed to systems and methods for performing multi-analyte testing. In some embodiments, an analyte testing device is provided that includes a plurality of modules, each module of the plurality of modules configured to perform one or more of a plurality of different analyte tests. Each module of the plurality of modules includes at least one sample port for receiving a blood sample, and at least one microprocessor configured to calculate an amount of one or more different analytes in the blood sample and prepare test results based on the calculated one or more different analytes. A base unit is provided having a plurality of receptacles configured to receive the plurality of modules such that an electrical connection is established between the base unit and the plurality of modules. Each module of the plurality of modules is configured to utilize the electrical connection to communicate the test results from each module of the plurality of modules to a processor of the base unit.

In some embodiments, at least one module of the plurality of modules is configured to perform at least one of a photochemical test on the blood sample and an electrochemical test on the blood sample.

In some embodiments, the plurality of modules includes a lipid module configured to perform a lipid panel. The lipid panel includes testing for LDL-Cholesterol, HDL-Cholesterol, total cholesterol, total triglycerides, Apolipoprotein B, and Lipoprotein (a). In some embodiments, the lipid module includes testing components for performing photometric tests for total cholesterol, total triglycerides, HDL-Cholesterol, and LDL-Cholesterol, and electrochemical tests for total cholesterol and total triglycerides.

In some embodiments, the plurality of modules includes a diabetic module configured to perform a diabetic panel. The diabetic panel includes testing for glucose, Glucose+HbA1c, Glucose+ketone, HbA1c+Hemoglobin, Glucose+Triglycerides, and Glucose+Lactate. In some embodiments, the diabetic module includes testing components for performing electrochemical tests for glucose, ketones, triglycerides, and lactate, and photometric tests for hemoglobin and A1C.

In some embodiments, the plurality of modules includes a renal module configured to perform a renal panel. The renal panel includes testing for sodium, Potassium, Creatinine, Creatinine+BUN, Uric Acid, Bicarbonate, Vitamin D, and Ferritin.

In some embodiments, the plurality of modules includes a liver module configured to perform a liver panel. The liver panel includes testing for ALT/AST, Albumin, Total Bilirubin, Cystatin C, and Apolipoprotein B.

In some embodiments, the analyte testing device further includes at least one panel interface associated with each module, wherein the base unit is configured to provide power and communications to each module through the at least one panel interface. In some embodiments, the at least one microprocessor is configured to communicate test results using the at least one panel interface.

In some embodiments, an analyte testing device is provided that includes a plurality of modules, each module of the plurality of modules configured to perform one or more of a plurality of different analyte tests. Each module of the plurality of modules includes at least one sample port for receiving a blood sample. A base unit is provided having a plurality of receptacles configured to receive the plurality of modules such that an electrical connection is established between the base unit and the plurality of modules. Each module of the plurality of modules is configured to utilize the electrical connection to communicate a plurality of test results associated with the plurality of different analyte tests from each module of the plurality of modules to the base unit. At least one microprocessor is provided that is configured to calculate an amount of one or more different analytes in the blood sample, and prepare test results based on the calculated one or more different analytes. A display is configured to display test results from the at least one microprocessor.

In some embodiments, the at least one microprocessor is located in the plurality of modules. In some embodiments, the at least one microprocessor is located in the base unit. In some embodiments, the display is integrated into the base unit. In some embodiments, the display is a remote device in electrical communication with the base unit.

In some embodiments, at least one module of the plurality of modules is configured to perform at least one of a photochemical test on the blood sample and an electrochemical test on the blood sample.

In some embodiments, a method of analyte testing is provided that includes providing a first sample to a first module of a plurality of modules and providing a second sample to a second module of the plurality of modules. Each of the first module and the second module includes at least one sample port for receiving a sample, and at least one microprocessor configured to calculate an amount of one or more different analytes in the sample, and prepare test results based on the calculated one or more different analytes. The method further includes performing, using the first module, a first plurality of tests on the first sample to determine a first set of test results, and performing, using the second module, a second plurality of tests on the second sample to determine a second set of test results. The method further includes communicating the first set of test results and the second set of test results to a base unit. The base unit has a plurality of receptacles configured to receive the plurality of modules such that an electrical connection is established between the base unit and the plurality of modules. The method further includes displaying the first set of test results and the second set of test results on a display device in electrical communication with the base unit.

In some embodiments, the method further includes calculating, with a first microprocessor in the first module, the first set of test results using a measured plurality of different analytes in the first sample, calculating, with a second microprocessor in the second module, the second set of test results using a measured plurality of different analytes in the second sample, and communicating the first set of test results and the second set of test results to the base unit.

In some embodiments, at least one module of the plurality of modules performs at least one of a photochemical test on the sample and an electrochemical test on the sample.

In some embodiments, the plurality of modules includes a lipid module to perform a lipid panel, the lipid panel including testing for LDL-Cholesterol, HDL-Cholesterol, total cholesterol, total triglycerides, Apolipoprotein B, and Lipoprotein (a). In some embodiments, the plurality of modules includes a diabetic module to perform a diabetic panel, the diabetic panel including testing for glucose, Glucose+HbA1c, Glucose+ketone, HbA1c+Hemoglobin, Glucose+Triglycerides, and Glucose+Lactate. In some embodiments, the plurality of modules includes a renal module to perform a renal panel, the renal panel including testing for sodium, Potassium, Creatinine, Creatinine+BUN, Uric Acid, Bicarbonate, Vitamin D, and Ferritin. In some embodiments, the plurality of modules includes a liver module to perform a liver panel, the liver panel including testing for ALT/AST, Albumin, Total Bilirubin, Cystatin C, and Apolipoprotein B.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 illustrates an exemplary embodiment of testing device comprising a plurality of analyte testing modules;

FIG. 2 illustrates the exemplary embodiment of the testing device of FIG. 1 with the testing modules separate from the testing device;

FIGS. 3A, 3B, and 3C illustrate an exemplary embodiment of a testing device with a plurality of analyte modules inserted therein and a display/user interface attached thereto;

FIGS. 4A, 4B, 4C, and 4D illustrate an exemplary embodiment of a testing device and an integrated display/user interface;

FIGS. 5A, 5B, 5C, and 5D illustrate an exemplary embodiment of a testing device having a display/user interface in the form of a remote external device;

FIG. 6 illustrates an exemplary embodiment of internal components of a module;

FIG. 7 illustrates an exemplary embodiment of internal components of a module;

FIG. 8 illustrates an exemplary embodiment of internal components of a module;

FIG. 9A illustrates an exemplary embodiment of internal components of a module having multiple sample ports of varying sizes;

FIG. 9B illustrates an exemplary embodiment of internal components of a module with multiple sample ports and AFEs;

FIG. 9C illustrates an exemplary embodiment of internal components of a module with multiple sample ports;

FIG. 10 illustrates an exemplary module connector pinout;

FIG. 11 illustrates exemplary test strip embodiments;

FIGS. 12A and 12B illustrates exemplary assay panels;

FIG. 13 illustrates an exemplary embodiment of a testing device with module for performing a lipid panel and a diabetic panel;

FIG. 14 illustrates exemplary test strips for use in a module of a testing device;

FIGS. 15A, 15B, and 15C. illustrate exemplary embodiments of modules that utilize different testing modalities; and

FIG. 16 illustrates an exemplary computer system suitable for use in connection with the systems and methods of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In particular, the systems and methods described herein are provided to test for multiple analytes using a modular testing device.

In some embodiments, a modular testing device is in the form of a multi-analyte panel test that can be used to perform any test or measurement. For example, a module of the modular testing device can be used to perform blood testing, urine testing, and/or any other diagnostic or analyte test. Thus, multiple types of testing can co-exist on a single device. The modules of the modular testing device can utilize existing tests, test panels, chemistries, and test strip technologies, in addition to new tests and test panels developed for use with the modular testing device. In some embodiments, legacy tests can be converted from optical to electrochemical to be used with the modular testing device.

A module-based system is also not tied to a single test strip technology or method. For example, one module could be electrochemical, whereas another module could be optical. Test strips can be different sizes, different blood volumes, and/or different technologies. The system allows for disparate technologies to co-exist in a single device, because each module can be custom designed for any necessary test. In some embodiments, the system can be used to perform multiple tests for a single patient at a time.

The module-based system is differentiated from other laboratory equipment and can keep costs low while having a high-performance system. In some embodiments, a hand-held portable device can be provided that utilizes one or more modules to extend the capabilities of the device to include whatever analyte tests are required, without needing to change the base of the device. Since the modules are user-changeable, there is no need for on-site service technicians if a module stops working. The module can simply be replaced by the health care provider. The devices could be shipped to customers pre-configured with a set of modules, or a base device can be shipped to customers and the customer installs whichever modules suit them best. The self-configuring aspect of this device allows for flexibility for the tests performed. In some embodiments, the users can include health care professionals, such as nurses, doctors, and phlebotomists. In some embodiments, the system can be located in clinics, HCP Offices, dialysis centers, pharmacy health club clinics, health fairs and health screenings. In some embodiments, the system can be portable and/or battery powered, approximately the size of a creatinine device.

In some embodiments, the system can be used in a laboratory, clinical or pharmacy setting, and can be run by health care professionals.

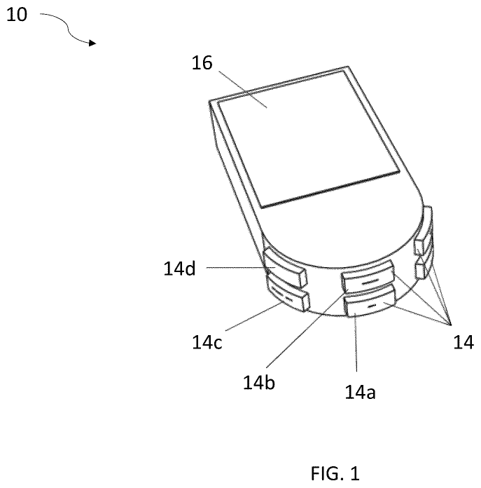

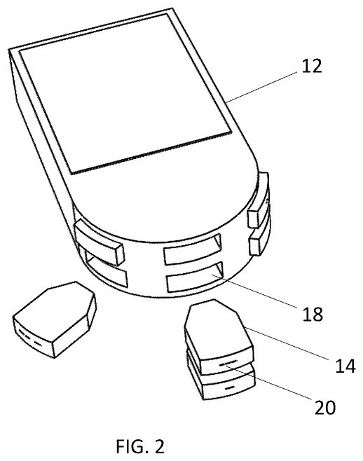

In some embodiments, as shown in FIGS. 1-2, the testing device 10 (e.g., a modular testing device) is configured to test for a plurality of analytes. In some embodiments, the testing device 10 includes a base unit 12 that includes a plurality of receptacles or module ports 18 formed in the base unit 12 of the testing device. Each module port 18 is configured to accept one or more modules 14 (e.g., test modules). In some embodiments, the module ports 18 are configured to be a standard receiving port to be able to accept a wide variety of modules. For example, the module ports 18 in the testing device can be of a standard shape and size and include an electrical connection therein for connecting with and communicating with any module when the module is inserted therein. In some embodiments, the module ports 18 can vary in size, shape, and/or electrical communication modality depending on variations in the modules themselves. It will be understood that the base unit 12 can be configured to accept any number of modules required depending on the use of the base unit. In some embodiments, the modules include pluggable modules. Pluggable modules include modules that are configured to plug into a receptacle or other receiver.

Each module 14 (e.g., a test module) is configured to run one or more tests, such as analyte tests, using any testing modality, including but not limited to an electrochemical testing modality or other sensor testing modality, such as optical, RF, IR, a pre-heater, etc. Each module can perform one or more different testing modalities simultaneously when a sample is inserted into a sample port 20 of the module. In some embodiments, each module contains one or more sample ports that are configured to accept an analyte sample or other device or sensor that includes the sample using a variety of modalities. For example, testing modalities that can be inserted in the modules can include analyte test strips. Each type of module is designed to measure one or more analytes via interface with a sensor, for example, a test strip. In some embodiments, the sample port can vary in size depending on the type of sample or test strip that is received by the modules, as shown by modules 14a, 14b in FIG. 1. In some embodiments, a module can include a sample port that is configured to receive a double-strip test (e.g., a test that requires at least two test strips). In some embodiments, a module can include more than one sample port for accepting more than analyte sample or test strip therein. For example, a module, such as module 14c shown in FIG. 1, can include a first port 22 and a second port 24 for receiving a first test strip and a second test strip can be used such that the module can perform two different testing modalities. Thus, the first test strip can be inserted into the first port 22 to perform, for example, an electrochemical test, and the second test strip can be inserted into the second port 24 to perform, for example, a photometric test. It will be understood that each module can include any number of ports to accept test samples, and the number of ports can be related to the types of tests performed by the module. In some embodiments, a module can be in the form of a blank module, such as module 14d shown in FIG. 1, i.e., a module that is not configured to measure an analyte. For example, a blank module 14d can be without a sample port. This can allow for the filing of a module port in the base unit that is not needed to run a specific panel of tests.

As shown in FIG. 2, the modules 14 can be removed from the module ports 18 of the testing device. As also shown in FIG. 2, the modules can be removed from a first receptacle or module port of the testing device and reinserted into a second receptacle or module port of the same testing device. For example, in some embodiments, a first module can be in a first receptacle during a first configuration of a testing device, and the first module can be in a second receptacle during a second configuration of a testing device. The configuration of the modules in the respective receptacles can be varied based on the test that is being administered, among other reasons. In some embodiments, the orientation of the modules relative to the receptacle can be changed. For example, in some embodiments, a module can be removed from a receptacle, rotated 180 degrees and reinserted into the same receptacle or a different receptacle. The orientation of the modules in the respective receptacles can be altered to more effectively space the modules, among other reasons. For example, if a module has a large extension that protrudes disproportionately from a certain side of the module, the module can be inserted into a receptacle such that the large extension does not interfere with the insertion of other modules.

While FIGS. 1-2 provide examples of testing devices with six module ports that can accommodate (e.g., accept) six modules, it is anticipated that testing devices can be produced with any number of receptacles that can accommodate an equal number of modules. For example, it is anticipated that a testing device can be created with ten receptacles that can accommodate ten modules. It is also anticipated that a single receptacle can be configured to accept more than one module. It will be understood that the base unit can be configured to accept any number of modules, related to the types of tests to be performed.

In some embodiments, the base unit of the testing device is configured to receive test results from the one or more modules. For example, each module plugged into the base unit is configured to communicate an amount and/or concentration of the different analytes for each test performed by each of the modules. The modules can also communicate additional test results related to the analytes that are calculated or determined by the modules. The information received from the modules by the base unit can also be stored in various types of storage in the base unit. In some embodiments, the base unit can include a processor and/or one or more storage devices to achieve this functionality.

In some embodiments, the base unit of the testing device is configured to communicate with a display or user interface. The display is configured to display information communicated from the one or more modules docked in the base unit. In some embodiments, the one or more modules are configured to communicate with the base unit through the electrical connection established between the module and a connector inside the module port. The display or user interface can be used to display results of the tests performed by the one or more modules. In some embodiments, the one or more modules are configured to communicate with the base unit to display information to the user. For example, directions for preparing a sample for testing or information about how to use a module can be displayed to the user. The display can be used to display any information relevant to the use of the testing device. For example, the display can display information about a patient (e.g., a patient's medical history, treatment history, identifying information, etc.), test results (e.g., results from the current testing, results from a previous test, test results from multiple tests, etc.), treatment instructions (e.g., how to administer the various tests, how frequently the tests should be administered, etc.), and other relevant information.

In some embodiments, a module can communicate display instructions that include instructions for displaying test results to the testing device. In some embodiments, a module can also communicate instructions to a user relating to use of the module and steps for performing the analyte test associated with the module. For example, a COVID module can communicate self-described test steps that can include instructions for the user to pre-mix something and start a timer, then insert the test strip.

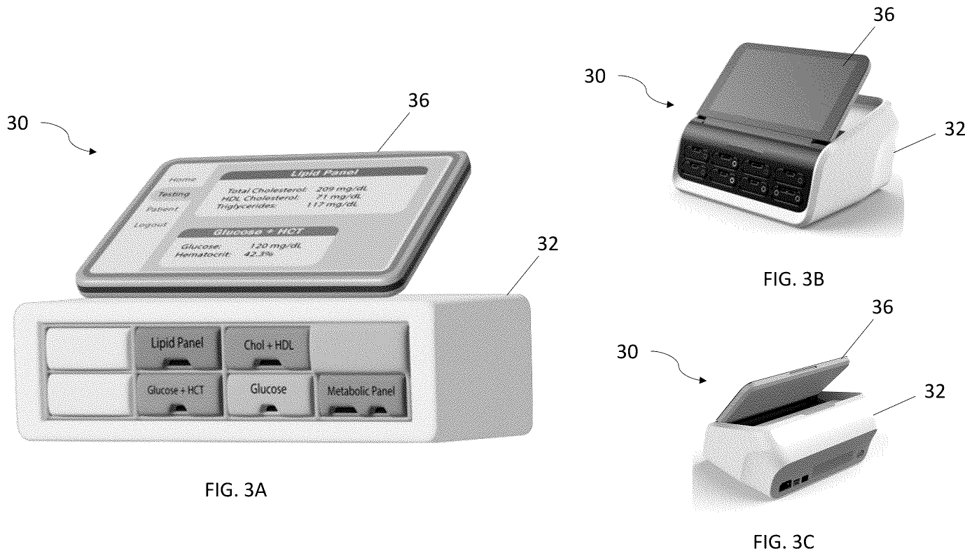

FIGS. 3A, 3B, and 3C illustrate an exemplary embodiment of a testing device 30 with a plurality of analyte modules inserted therein. As shown in FIG. 3A, the plurality of analyte modules can be inserted into a base unit 32 or module housing. The base unit 32 can contain one or more module ports. For example, as shown in FIGS. 3A and 3B, the base unit 32 includes eight module ports. In some embodiments, the module housing contains one or more spare modules. In some embodiments, a spare module or blank module can be inserted into a receptacle or module port instead of a module that can perform a test. For example, if a test or multiple tests, can be adequately performed using less than all of the receptacles or module ports, a spare module can be optionally inserted into a receptacle or module port instead of a test module. As can also be seen in FIG. 3A, the testing device can include a display 36 mounted on the base unit. The display can be connected to the other portions of the testing device such that the integrated display can be moved. For example, the display can be attached to the module housing and the display can rotate about an edge of the module housing to go from a compact position to an extended position. In some embodiments, the testing device has one or more components that are configured to store information. For example, the testing device can be configured to store information about a patient, test results, treatment instructions, and other relevant information.

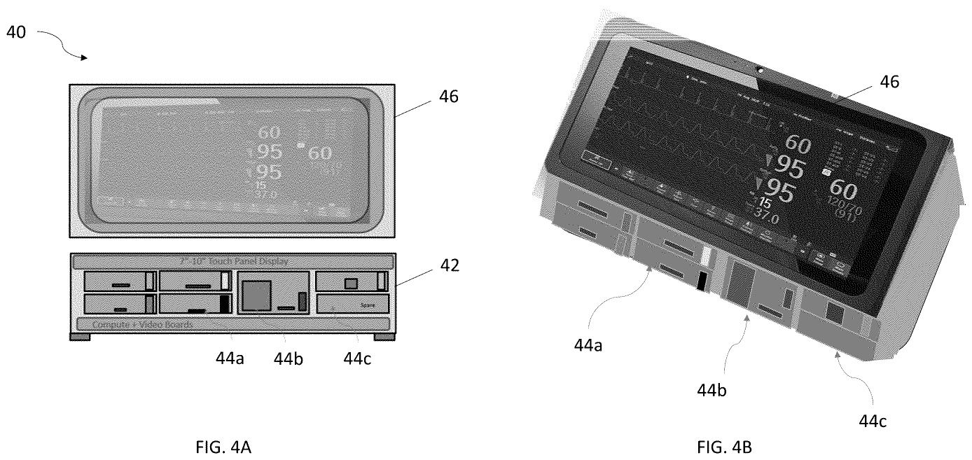

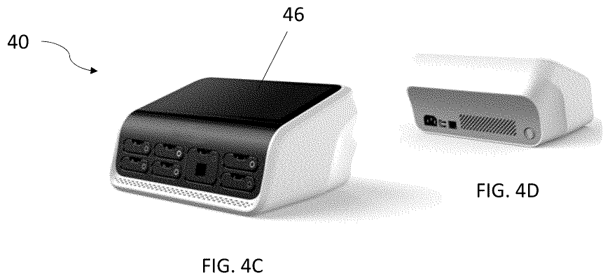

FIGS. 4A, 4B, 4C, and 4D illustrate an exemplary embodiment of a testing device 40 having an integrated display 46. The testing device 40 includes a base unit 42. As shown, the base unit 42 includes 7 module ports, with five of those module ports 44a for receiving a single module, one dual slot module port 44b, and a module port 44c configured to receive a spare or blank module. It will be understood that the base unit can include any number of module ports with any type of configuration. In some embodiments, the display can include a touchscreen to allow a user to interact with the results from the various testing done by the modules in the testing device. In some embodiments, an external controller can be used to allow a user to interact with the results from the various testing done by the modules in the testing device. In some embodiments, an external controller may include a keyboard, a mouse, a wireless remote, and/or a wired remote, among other devices. In some embodiments, the modules in the testing device can communicate test results, either wirelessly or through a wired communication, to the display and/or other commuting devices.

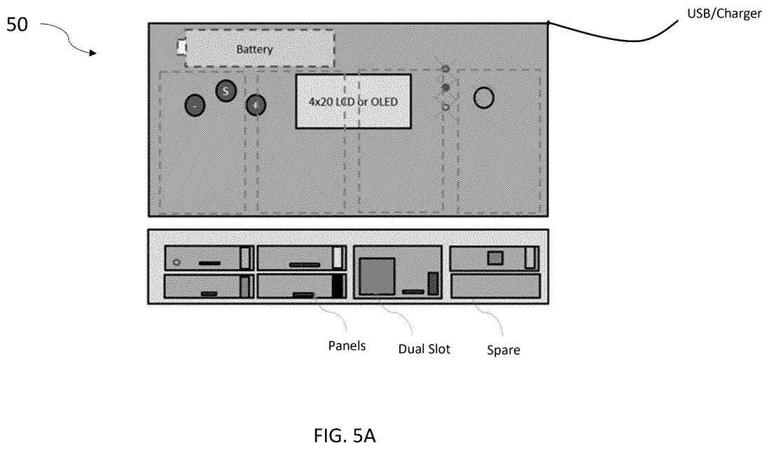

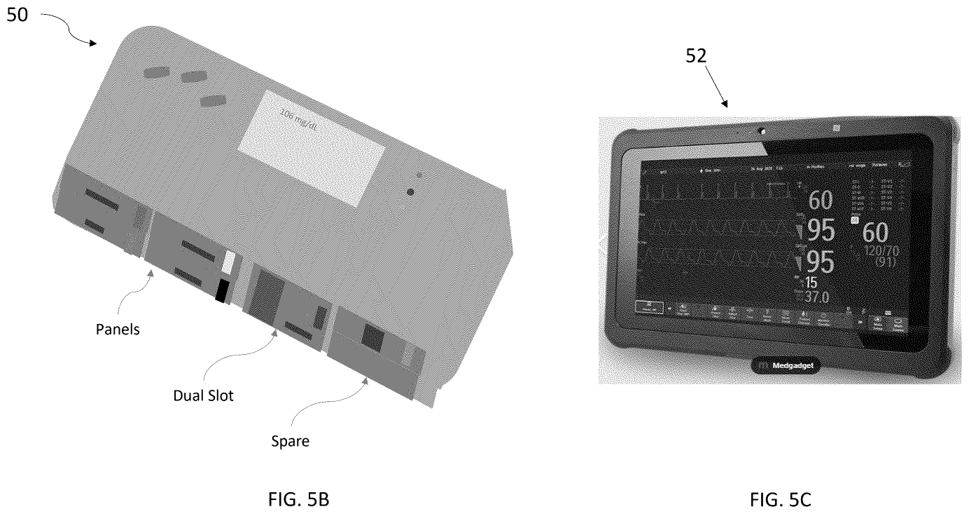

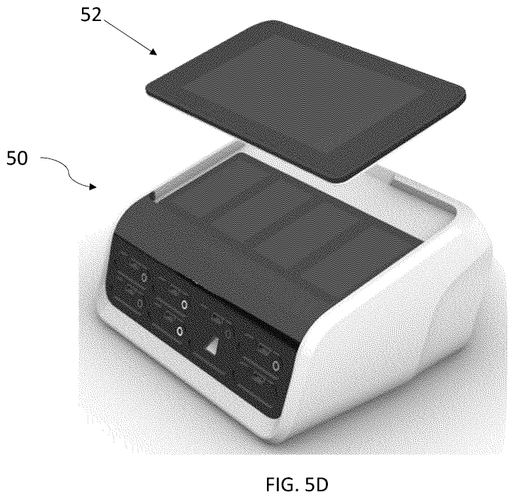

FIGS. 5A, 5B, 5C, and 5D illustrate an exemplary embodiment of a testing device 50 in communication with a remote external computing device 52, such as a computer, a smart phone, a tablet, or other external processor rather than an integrated display/touchscreen. In some examples, the testing device is in communication with a remote external device that is being monitored by a third party. For example, in some embodiments, a patient can administer a test using a testing device and a medical professional can see the results of the test on a remote external device. In some embodiments, the remote external device may have a display screen, a battery, user control aspects, and a wired connection configured to connect the remote external device to a communication source (e.g., a USB source) and/or a charger. In some embodiments, the user control aspects may enable the user to control the testing device. In some embodiments, the user control aspects may include one or more buttons, one or more analog sticks, and/or other apparatus that may enable a user to control the testing device.

In some embodiments, a system can be used to support up to multiple modules that have simultaneous measurement capability. In some embodiments, the system can include two to ten modules. In some embodiments, the system may include up to 8 modules. There can be a remote tablet/phone user interface (“UI”) option, and remote software update for modules and communication processors. The system can include an option for battery power. Modules can be placed in any open receptacle or module port in the system, so the modules are interchangeable relative to the receptacles or module ports.

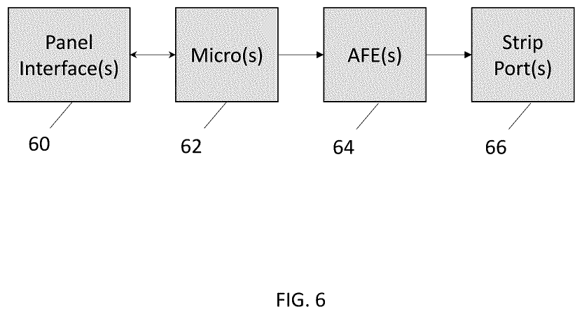

In some embodiments, each module can include all the necessary components to electrically stimulate the sensor (if required), read the sensor, collect measurements, execute an algorithm, and/or calculate one or more results based on the analyte sample associated with each module. Thus, all the necessary hardware and software to run each test can be performed by each module without needed interference from the base unit. Thus, in some embodiments, the module functions as its own self-contained sensor reading measurement device. For example, a module will include a panel interface 60 to provide power and communications between the module and the display interface through the electrical connection in a module port, a microcontroller 62 for processing the information from analyte test with internal or external analog front end (AFE) 64 and a sample port or an electrochemical strip port connector interface 66, as shown in FIG. 6. The sample can be introduced into the module using the sample port or the strip port such that the testing indicated by the module can be performed on the sample. The signal produced can be converted to from an analog signal to a digital signal using the AFE, which communicates the digital signal to the microprocessor. The AFE can be electrochemical, optical, RF, magnetic, nuclear, or other sensor technology The microprocessor can include functionality to process the signal to produce test results that can be communicated to the user and displayed on a display, touchscreen, or other remote device as described above.

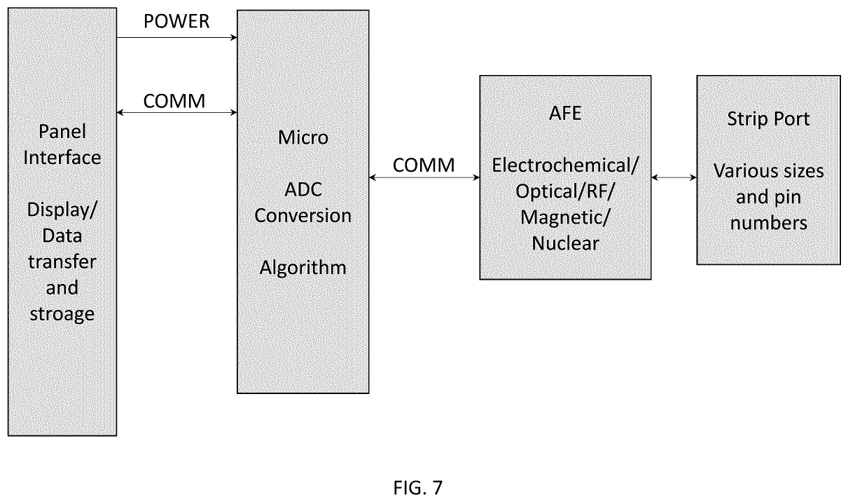

FIG. 7 illustrates a more detailed view of the components inside a self-contained module. The base unit of the testing device is configured to provide power and communications to the sensor modules through the panel interface. All the sensor processing is done on the module and the end results are communicated back to the base unit for display, storage, and/or transfer via WiFI to the internet or Bluetooth to remote device, such as a tablet computer, or smart phone.

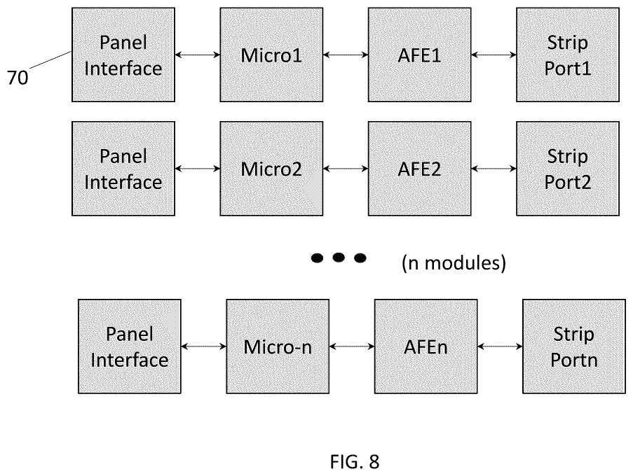

In some embodiments, for n modules (n>1), there may be different analog front ends (AFEs) and different strip ports and different microcontrollers, as shown in the embodiment in FIG. 8. In some embodiments, the panel interface can be the same for all sensor modules. The AFEs can be electrochemical, optical, RF, magnetic, nuclear, or other sensor technology. The strip ports may vary in size, number of connections, and interface to AFE. The microcontroller may vary in memory, speed, size, pinout, package, manufacturer, etc. to suite to the needs of the sensor processing. For example, as shown in FIG. 8, there can be a plurality of modules, each with their own strip port, AFE, and microcontroller. The panel interface 70 can be shared with all of the plurality of modules, or the system can include separate interfaces for each of the plurality of modules.

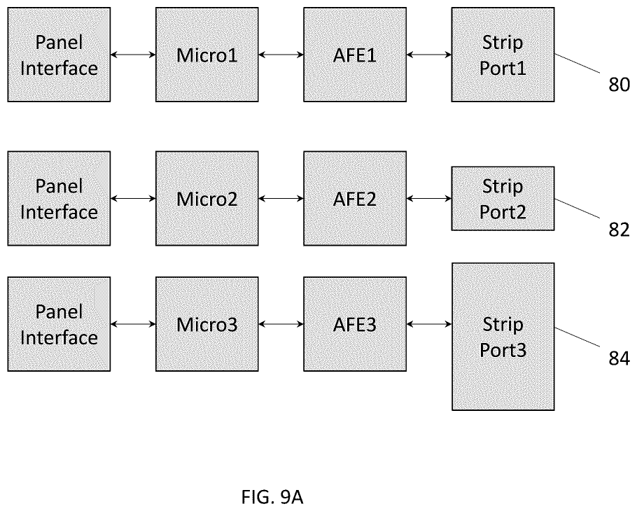

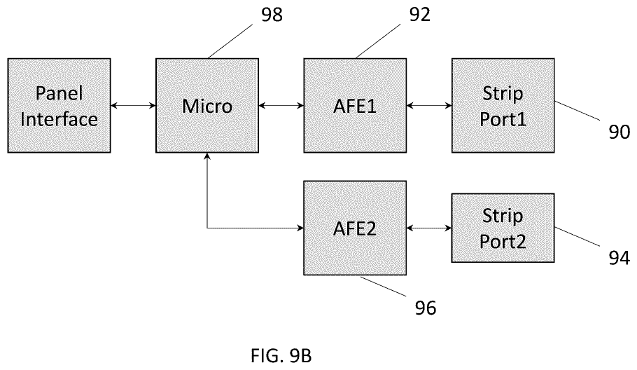

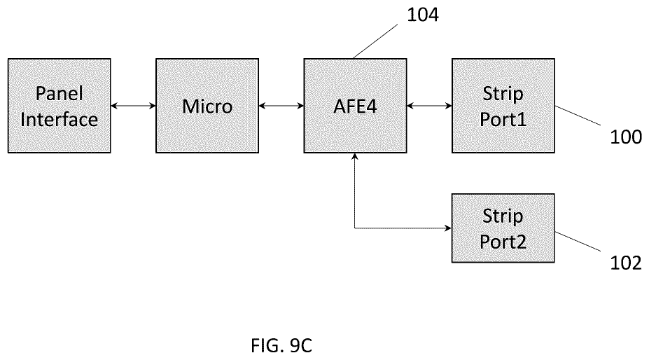

In some embodiments, a module can have multiple strip ports and AFEs. FIG. 9A illustrates an embodiment of a module with multiple strip ports that are different sizes (for example, strip ports 80, 82, 84) such that a module can accommodate any type and/or size of test strip. It will be understood that different modules can also have different types and/or size of ports so different modules can accommodate different types and/or sizes of test strips. FIG. 9B illustrates an embodiment of a module that includes a first strip port 90 in communication with a first AFE 92 and a second strip port 94 in communication with a second AFE 96. Both the first AFE and the second AFE are in communication with a single microcontroller 98. FIG. 9C illustrates an embodiment of a module that includes a first strip port 100 and a second strip port 102 in communication with a single AFE 104.

Each module can also be in electrical communication with the base unit. For example, the module can handshake with the base unit over a communication channel to communicate information to the base unit to communicate test results to the base unit that can be used for user interface activity. This allows the base unit to display the information to a user relating to one or more results associated with the analyte tests/measurements performed by the module.

In some embodiment, a pregnancy module can communicate a self-described result to the base unit that is non-numeric, for example, to display positive or negative.

In some embodiments, the base unit is configured to coordinate module testing, handle power conversion and battery charging and backup, and/or communicate with a user, wired or wirelessly.

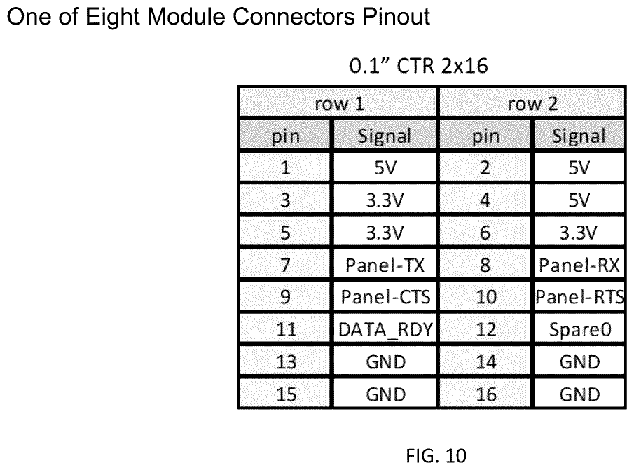

In some embodiments, power is supplied to the module from the base unit. In some embodiments, for connection to a receptacle in the base unit, each module pinout can be identical, and can include power, ground, communications TX/RX and handshaking and well as a data ready input and a spare (see exemplary embodiment shown in FIG. 10).

The diagnostic testing device described herein can be used to test for a variety of testing applications in different industries. Alternate embodiments could include environmental diagnostic testing, such as water testing (i.e., pool water or drinking water), testing for the food/beverage industry, and medical diagnostic testing, such as or virus/antibody testing, as well as health and fitness markers such as pH, and blood-sugar stabilization. In some embodiments, the modules contain software with self-describing and identifying information. In this way, the base unit can read and communicate with a new module and understand the type of test, unit of measure, number of analytes, user interface features required, and/or any other relevant information the base unit would need from the module. This allows for increased flexibility with the testing device as the testing device can use any module designed for any test without having to alter the base unit of the testing device.

For example, a module can be configured to perform a multiple-analyte test, for example, using a single test strip. For example, a module can be configured to perform tests for glucose and hematocrit using a single test strip. The module that performs more than one test will need to describe that to the base unit. For example, the module can communicate the dual-analyte information even though there is only a single test strip. The module can also describe the steps of the testing sequence, which can include application of the sample, sample filled, a test count down, and display of results. In this example, a result description will include mg/dL for glucose and a percentage for hematocrit, along with the appropriate number of decimal points to display. Label descriptions can also be conveyed to the base unit. For example, the module can include information such that the display can shown “Hematocrit” as “HCT.”

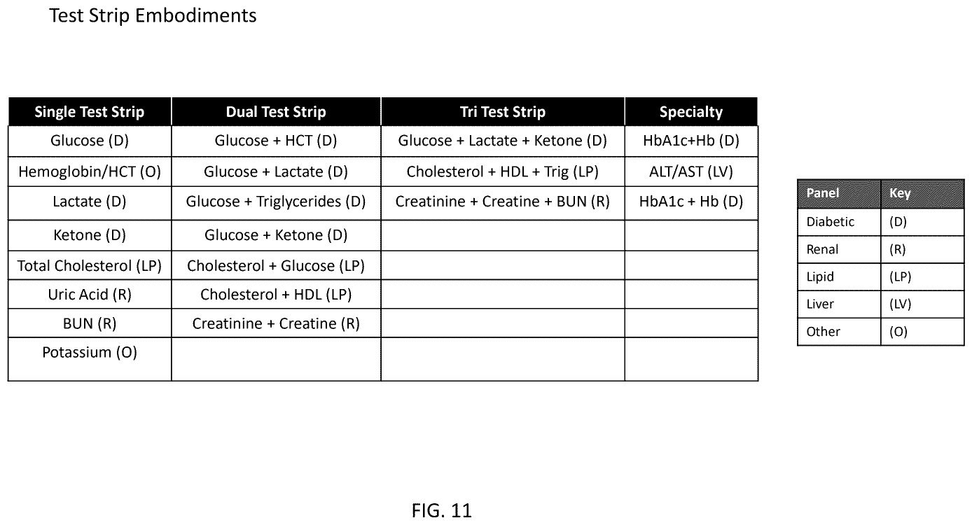

Various test strip embodiments can be used with the modules in the testing device. For example, FIG. 11 illustrates various embodiments of test strips that can be used to perform a variety of analyte tests and can be used with the modules of the testing device. In some embodiments, a test strip can be used to test for a single analyte. Exemplary test strips that can test for a single analyte include tests strips for glucose, hemoglobin/hematocrit (“HCT”), lactate, ketone, total cholesterol, uric acid, blood urea nitrogen (“BUN”), and potassium, among others. In some embodiments, a test strip can be used to test for two analytes. Exemplary test strips that can be used to test for two analytes include tests strips for glucose and HCT, glucose and lactate, glucose and triglycerides, glucose and ketone, cholesterol and glucose, cholesterol and high-density lipoprotein (“HDL”), and creatinine and creatine, among other combinations. In some embodiments, a test strip can be used to test for three analytes. Exemplary test strips that can be used to test for three analytes include tests strips for glucose, lactate, and ketone, cholesterol, HDL, and triglycerides, and creatinine, creatine, and BUN, among other combinations. In some embodiments, a test strip can be used to test for a plurality of analytes. In some embodiments, the test can be a test that is associated with a diabetic test, a renal test, a lipid test, or a liver test, among others. In some embodiments, a diabetic test may include testing for glucose, lactate, ketone, HCT, triglycerides, and HbA1c+hemoglobin (“Hb”), among other tests. In some embodiments, a renal test may include testing for uric acid, BUN, creatine, and creatinine, among other tests. In some embodiments, a lipid test may include testing for total cholesterol, glucose, HDL, and triglycerides, among other tests. In some embodiments, liver tests may include testing for alanine transaminase (“ALT”)/aspartate transaminase (“AST”), among other tests. In some embodiments, other tests may include testing for hemoglobin/HCT and potassium, among other tests. In some embodiments, the test strip can be used for a specialty test. In some embodiments, specialty tests include HbA1c+Hb, and ALT/AST.

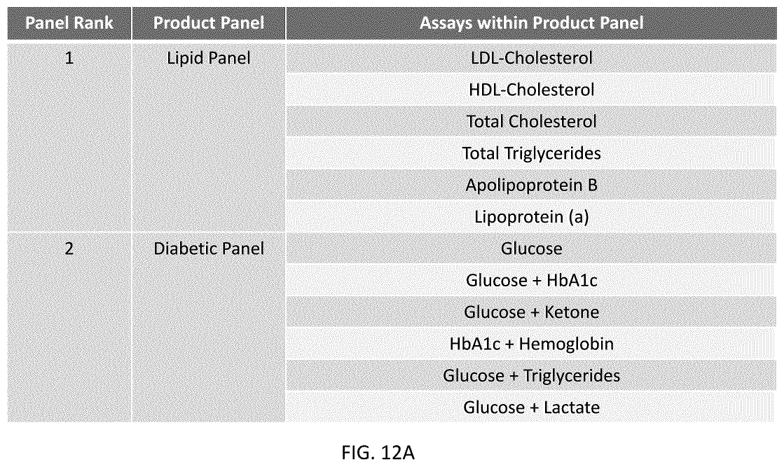

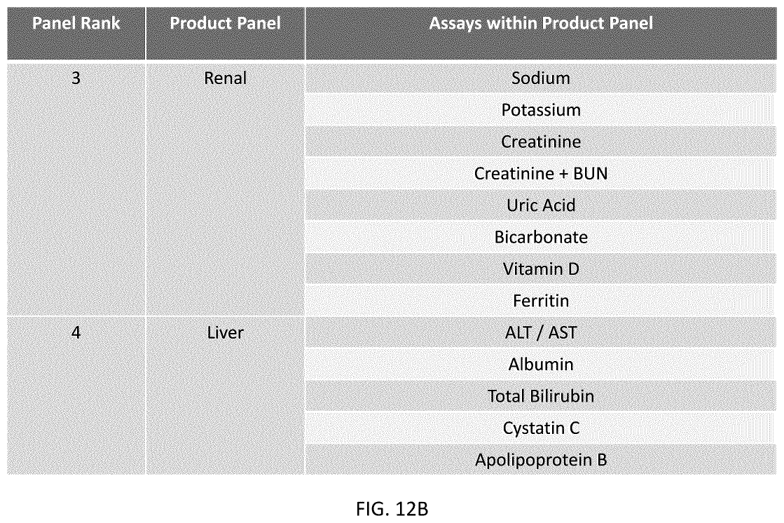

FIGS. 12A and 12B illustrate exemplary embodiments of assay panels that can be used to test for a plurality of analytes simultaneously. For example, a lipid panel can test for LDL-Cholesterol, HDL-Cholesterol, total cholesterol, total triglycerides, Apolipoprotein B, and Lipoprotein (a). The accuracy requirements for each analyte in the assay can be as follows:
LDL-Cholesterol is ±20%.
HDL-Cholesterol is ±6 mg/dL or ±20%.
Total cholesterol is ±10%.
Total triglycerides is ±15%.
Apolipoprotein B is ±20%.
Lipoprotein (a) can vary.

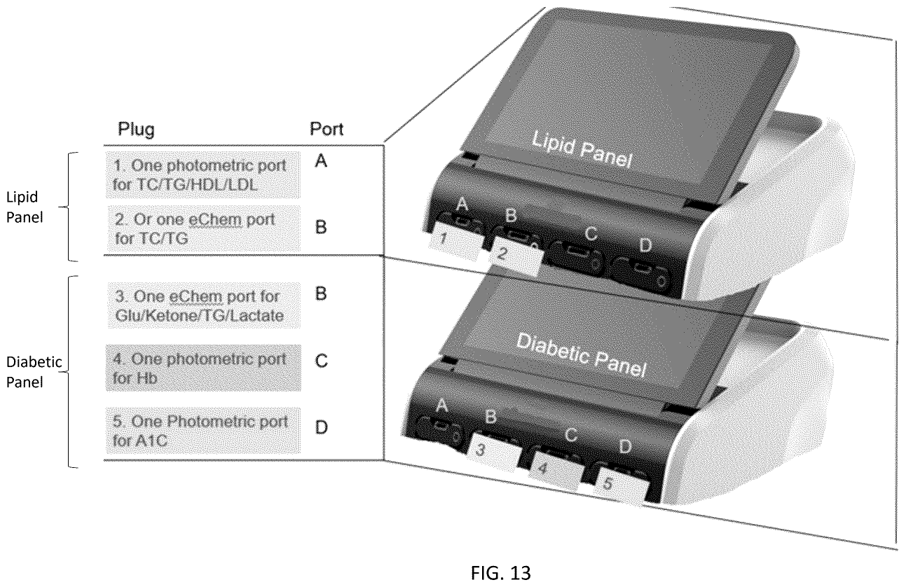

In some embodiments, multiple modules or multiple ports can be used to test for the analytes in the lipid panel. For example, as shown in FIG. 13, a photometric port for TC, TG, HDL, and LDL can be used, and an electrochemical port for TC and TG can be used.

A diabetic panel can test for glucose, Glucose+HbA1c, Glucose+ketone, HbA1c+Hemoglobin, Glucose+Triglycerides, and Glucose+Lactate. The accuracy requirements for each analyte in the assay can be as follows:
Glucose is ±6 mg/dL or ±8% (PT) or ±12% (POC).
Glucose+HbA1c is ±8% for A1c.
Glucose+ketone is ±15% for ketones.
HbA1c+Hemoglobin is ±4% for Hb.
Glucose+Triglycerides is ±15% for triglycerides.
Glucose+Lactate is ±15% for lactate.

In some embodiments, multiple modules or multiple ports can be used to test for the analytes in the diabetic panel. For example, as shown in FIG. 13, an electrochemical port for glucose, ketones, TG, and lactate can be used, a photometric port for Hb can be used, and a photometric port for A1C can be used.

A renal panel can test for sodium, Potassium, Creatinine, Creatinine+BUN, Uric Acid, Bicarbonate, Vitamin D, and Ferritin. The accuracy requirements for each analyte in the assay can be as follows:
Sodium is ±4 mmol/L.
Potassium is ±0.3 mmol/L.
Creatinine is ±0.2 mg/dL or ±10%.
Creatinine+BUN is ±2 mg/dL or ±9% for BUN.
Uric Acid is ±10%.
Bicarbonate is ±7%.
Vitamin D can vary.
Ferritin is ±20%.

In some embodiments, multiple modules or multiple ports can be used to test for the analytes in the renal panel. For example, an electrochemical port for creatinine can be used, an electrochemical port for UA can be used, and a photometric port for VD and Ferr can be used.

A liver panel can test for ALT/AST, Albumin, Total Bilirubin, Cystatin C, and Apolipoprotein B. The accuracy requirements for each analyte in the assay can be as follows:
ALT/AST ±6% U/L or ±15%.
Albumin is ±8%.
Total Bilirubin is ±20%.
Cystatin C is ±10%.
Apolipoprotein B Is ±20%.

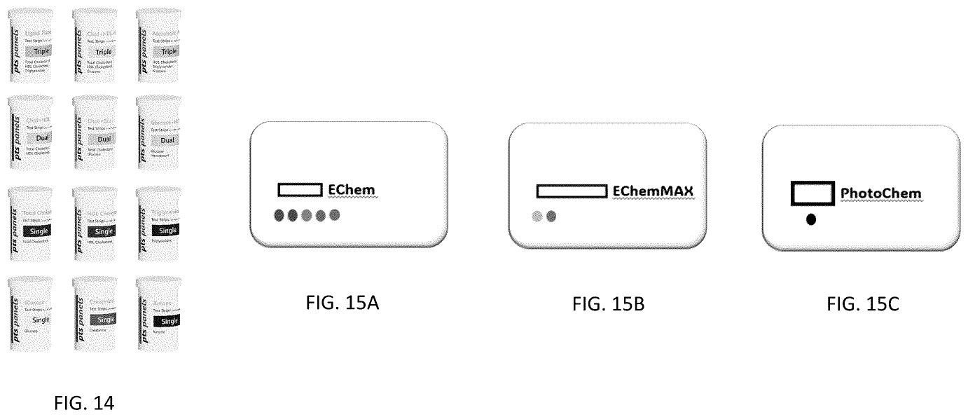

FIG. 14 illustrates containers of exemplary test strips for use in a module of a testing device. The containers of exemplary test strips can include one or more test strips. In some embodiments, as discussed in more detail below, the test strips can be used for measuring one or more analytes. In some embodiments, the containers can include indicia related to the type of tests that are performed by the test strips, such that the modules can be coded with those indicia for ease of coupling the correct test strips and modules (for example, the modules shown in FIGS. 15A, 15B, and 15C).

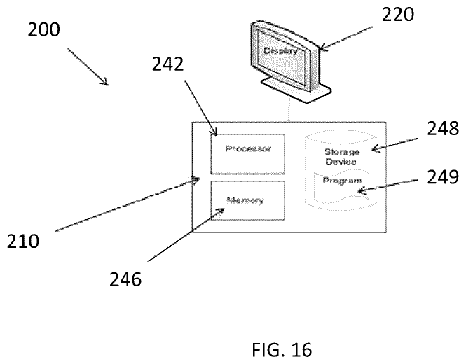

FIG. 16 shows, by way of example, a diagram of a typical processing architecture 200, which may be used in connection with the methods and systems of the present disclosure. A computer processing device 210 can be coupled to display 220 for graphical output. Processor 242 can be a computer processor 242 capable of executing software. Typical examples can be computer processors (such as Intel® or AMD® processors), ASICs, microprocessors, and the like. Processor 242 can be coupled to memory 246, which can be typically a volatile RAM memory for storing instructions and data while processor 242 executes. Processor 242 may also be coupled to storage device 348, which can be a non-volatile storage medium, such as a hard drive, FLASH drive, tape drive, DVDROM, or similar device. Although not shown, computer processing device 210 typically includes various forms of input and output. The I/O may include network adapters, USB adapters, Bluetooth radios, mice, keyboards, touchpads, displays, touch screens, LEDs, vibration devices, speakers, microphones, sensors, or any other input or output device for use with computer processing device 210. Processor 242 may also be coupled to other types of computer-readable media, including, but not limited to, an electronic, optical, magnetic, or other storage or transmission device capable of providing a processor, such as the processor 242, with computer-readable instructions. Various other forms of computer-readable media can transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript.

Program 249 can be a computer program or computer readable code containing instructions and/or data, and can be stored on storage device 248. The instructions may comprise code from any computer-programming language, including, for example, C, C++, C#, Visual Basic, Java, Python, Perl, and JavaScript. In a typical scenario, processor 342 may load some or all of the instructions and/or data of program 249 into memory 246 for execution. Program 249 can be any computer program or process including, but not limited to web browser, browser application, address registration process, application, or any other computer application or process. Program 349 may include various instructions and subroutines, which, when loaded into memory 246 and executed by processor 242 cause processor 242 to perform various operations, some or all of which may effectuate the methods for managing medical care disclosed herein. The program 249 may be stored on any type of non-transitory computer readable medium, such as, without limitation, hard drive, removable drive, CD, DVD or any other type of computer-readable media.

In some embodiments, the computer system may be programmed to perform the steps of the methods of the present disclosure and control various parts of the instant systems to perform necessary operation to achieve the methods of the present disclosure. In some embodiments, the processor in the one or more modules may be programed to receive analyte data to determine an amount and/or concentration of one or more different analytes in the sample. In some embodiments, the processor in the base unit may be programed to receive test results and/or analyte amounts and/or concentrations, store test results and/or analyte amounts and/or concentrations, and communicate test results and/or analyte amounts and/or concentrations to a display.

Various aspects of the examples described above can be used alone, in combination, or in a variety of arrangements not specifically discussed in the examples described in the foregoing and is therefore not limited in its application to the details and arrangement of components set forth in the foregoing description or illustrated in the drawings. For example, aspects described in one example can be combined in any manner with aspects described in other examples.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” or “example” is used herein to mean serving as an example, instance, or illustration. Any embodiment, implementation, process, feature, etc. described herein as exemplary or as an “example” should therefore be understood to be an illustrative example and should not be understood to be a preferred or advantageous example unless otherwise indicated.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one example, to A only (optionally including elements other than B); in another example, to B only (optionally including elements other than A); in yet another example, to both A and B (optionally including other elements); etc.

Having thus described several aspects of at least one example, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1. An analyte testing device, comprising:
a plurality of modules, each module of the plurality of modules configured to perform one or more of a plurality of different analyte tests, each module of the plurality of modules comprising:
at least one sample port for receiving a blood sample; and
at least one microprocessor configured to:
calculate an amount of one or more different analytes in the blood sample; and
prepare test results based on the calculated one or more different analytes; and
a base unit having a plurality of receptacles configured to receive the plurality of modules such that an electrical connection is established between the base unit and the plurality of modules, each module of the plurality of modules being configured to utilize the electrical connection to communicate the test results from each module of the plurality of modules to a processor of the base unit.

2. The analyte testing device of claim 1, wherein at least one module of the plurality of modules is configured to perform at least one of a photochemical test on the blood sample and an electrochemical test on the blood sample.

3. The analyte testing device of claim 1, wherein the plurality of modules comprises a lipid module configured to perform a lipid panel, the lipid panel including testing for LDL-Cholesterol, HDL-Cholesterol, total cholesterol, total triglycerides, Apolipoprotein B, and Lipoprotein (a).

4. The analyte testing device of claim 3, wherein the lipid module comprises testing components for performing photometric tests for total cholesterol, total triglycerides, HDL-Cholesterol, and LDL-Cholesterol, and electrochemical tests for total cholesterol and total triglycerides.

5. The analyte testing device of claim 1, wherein the plurality of modules comprises a diabetic module configured to perform a diabetic panel, the diabetic panel including testing for glucose, Glucose+HbA1c, Glucose+ketone, HbA1c+Hemoglobin, Glucose+Triglycerides, and Glucose+Lactate.

6. The analyte testing device of claim 5, wherein the diabetic module comprises testing components for performing electrochemical tests for glucose, ketones, triglycerides, and lactate, and photometric tests for hemoglobin and A1C.

7. The analyte testing device of claim 1, wherein the plurality of modules comprises a renal module configured to perform a renal panel, the renal panel including testing for sodium, Potassium, Creatinine, Creatinine+BUN, Uric Acid, Bicarbonate, Vitamin D, and Ferritin.

8. The analyte testing device of claim 1, wherein the plurality of modules comprises a liver module configured to perform a liver panel, the liver panel including testing for ALT/AST, Albumin, Total Bilirubin, Cystatin C, and Apolipoprotein B.

9. The analyte testing device of claim 1, further comprising at least one panel interface associated with each module, wherein the base unit is configured to provide power and communications to each module through the at least one panel interface.

10. The analyte testing device of claim 9, wherein the at least one microprocessor is configured to communicate test results using the at least one panel interface.

11. An analyte testing device, comprising:
a plurality of modules, each module of the plurality of modules configured to perform one or more of a plurality of different analyte tests, each module of the plurality of modules comprising at least one sample port for receiving a blood sample;
a base unit having a plurality of receptacles configured to receive the plurality of modules such that an electrical connection is established between the base unit and the plurality of modules, each module of the plurality of modules being configured to utilize the electrical connection to communicate a plurality of test results associated with the plurality of different analyte tests from each module of the plurality of modules to the base unit;
at least one microprocessor configured to:
calculate an amount of one or more different analytes in the blood sample; and
prepare test results based on the calculated one or more different analytes; and
a display configured to display test results from the at least one microprocessor.

12. The analyte testing device of claim 11, wherein the at least one microprocessor is located in the plurality of modules.

13. The analyte testing device of claim 11, wherein the at least one microprocessor is located in the base unit.

14. The analyte testing device of claim 11, wherein the display is integrated into the base unit.

15. The analyte testing device of claim 11, wherein the display is a remote device in electrical communication with the base unit.

16. The analyte testing device of claim 11, wherein at least one module of the plurality of modules is configured to perform at least one of a photochemical test on the blood sample and an electrochemical test on the blood sample.

17. A method of analyte testing, comprising:
providing a first sample to a first module of a plurality of modules, and providing a second sample to a second module of the plurality of modules, each of the first module and the second module comprising:
at least one sample port for receiving a sample; and
at least one microprocessor configured to calculate an amount of one or more different analytes in the sample, and prepare test results based on the calculated one or more different analytes;
performing, using the first module, a first plurality of tests on the first sample to determine a first set of test results;
performing, using the second module, a second plurality of tests on the second sample to determine a second set of test results;
communicating the first set of test results and the second set of test results to a base unit, the base unit having a plurality of receptacles configured to receive the plurality of modules such that an electrical connection is established between the base unit and the plurality of modules; and
displaying the first set of test results and the second set of test results on a display device in electrical communication with the base unit.

18. The method of claim 17, further comprising:
calculating, with a first microprocessor in the first module, the first set of test results using a measured plurality of different analytes in the first sample;
calculating, with a second microprocessor in the second module, the second set of test results using a measured plurality of different analytes in the second sample; and
communicating the first set of test results and the second set of test results to the base unit.

19. The method of claim 17, wherein at least one module of the plurality of modules performs at least one of a photochemical test on the sample and an electrochemical test on the sample.

20. The method of claim 17, wherein the plurality of modules comprises a lipid module to perform a lipid panel, the lipid panel including testing for LDL-Cholesterol, HDL-Cholesterol, total cholesterol, total triglycerides, Apolipoprotein B, and Lipoprotein (a).

21. The method of claim 17, wherein the plurality of modules comprises a diabetic module to perform a diabetic panel, the diabetic panel including testing for glucose, Glucose+HbA1c, Glucose+ketone, HbA1c+Hemoglobin, Glucose+Triglycerides, and Glucose+Lactate.

22. The method of claim 17, wherein the plurality of modules comprises a renal module to perform a renal panel, the renal panel including testing for sodium, Potassium, Creatinine, Creatinine+BUN, Uric Acid, Bicarbonate, Vitamin D, and Ferritin.

23. The method of claim 17, wherein the plurality of modules comprises a liver module to perform a liver panel, the liver panel including testing for ALT/AST, Albumin, Total Bilirubin, Cystatin C, and Apolipoprotein B.
